(19) United States
(12) Patent Application Publication
(10) Pub. No.: US 2020/0038029 A1
(43) Pub. Date: Feb. 6, 2020

(54) SURGICAL FASTENER AND APPARATUS

(71) Applicant: IMPERIAL INNOVATIONS LIMITED, London, Greater London (GB)

(72) Inventors: Mikael Sodergren, London (GB); Christopher Payne, Cambridge, MA (US)

(21) Appl. No.: 16/341,400

(22) PCT Filed: Oct. 10, 2017
(86) PCT No.: PCT/GB2017/053057

§ 371 Date: Apr. 11, 2019

(30) Foreign Application Priority Data
Oct. 12, 2016 (GB) 1617292.6

(51) Int. Cl. A61B 17/122; A61B 17/128

(52) U.S. Cl. CPC A61B 17/122; A61B 2017/00628; A61B 17/1285

(57) ABSTRACT

A surgical fastener having a first part and a second part, each of the first and second parts comprising a clamping bar and a centrally-positioned perpendicular bracing bar extending therefrom, the clamping bar and bracing bar together defining a “T”-shape, wherein the surgical fastener further comprises at least one bracing element connecting the bracing bar of each of the first and second parts. A surgical apparatus for the application of a fastener to tissue comprising a handle assembly, a shaft extending distally from the handle assembly, a drive assembly, an articulation assembly and a jaw assembly mounted adjacent a distal end of the shaft comprising fixed support jaw and a clamping jaw, at least one of the jaws comprises a rotatable fastener support defining a T-shaped recess therein for receiving a surgical fastener, wherein the articulation assembly is operable to rotate the fastener support and the drive assembly is operable by the handle assembly to move the clamping jaw in and out of a closed configuration with respect to the support jaw.

### FIELD OF THE INVENTION

This invention relates to surgical fasteners and to a surgical fastener apparatus for applying one or more surgical fasteners to body tissue.

### BACKGROUND OF THE INVENTION

Surgical procedures requiring cutting of tissue often result in bleeding at the site. Techniques have been adapted to control bleeding, for example, suturing, applying fasteners to blood vessels and using surgical fasteners or staples, as well as electro-cautery. Often a combination of these techniques is needed to control bleeding satisfactorily.

Laparoscopic surgery is a type of surgery in which clinicians operate through small incisions made in to the patient's abdomen. Sealed cannulas known as a trocar ports are placed through the incisions which allows insufflation of the abdominal cavity so as to create a surgical workspace. Long, slender laparoscopic instrumentation can be passed through the sealed cannulas and interchanged during the surgical procedure, thus avoiding large incisions to be made in to the patient. During laparoscopic surgery there is often a requirement to both cut and clamp folds of tissue together or to occlude the flow of blood or other fluids through anatomical lumens. Surgical fasteners are typically used to perform this action. These can be plastic fasteners with an interlock mechanism that clamp, for example as in U.S. Pat. Nos. 6,716,226, 4,512,345, US 2004/0193188 and US 2004/0193189. Surgical fasteners can also be made from a ductile material that plastically deform under loading so as to clamp the tissue together, such fasteners are described in U.S. Pat. No. 5,084,057. Traditional elastic fasteners are described in U.S. Pat. No. 5,053,045.

The surgical fastener should function to occlude the lumen which it is intended to clamp off. Further, the fastener must therefore not slip with respect to the lumen and the fastener must be of sufficient length to fully occlude the vessel. Other important characteristics include being devoid of sharp edges and excessive force so as not to cause perforation of the tissue being clamped off. For example: a laparoscopic surgical fastener may have a series of transverse or lateral notches in the clamping region of the fastener in order to prevent longitudinal and lateral slippage with respect to the lumen as found in U.S. Pat. No. 4,844,066. In this design, the tips of the fastener legs are brought together before the fastener is fully clamped, this ensures that the vessel lumen is fully trapped between the fasteners and ensures that the lumen becomes fully occluded after fastener has been clamped. Other designs include an elastic-based spring design in which the fastener has a planar configuration in which two flexures are used to trap the vessel as described in U.S. Pat. No. 8,372,095.

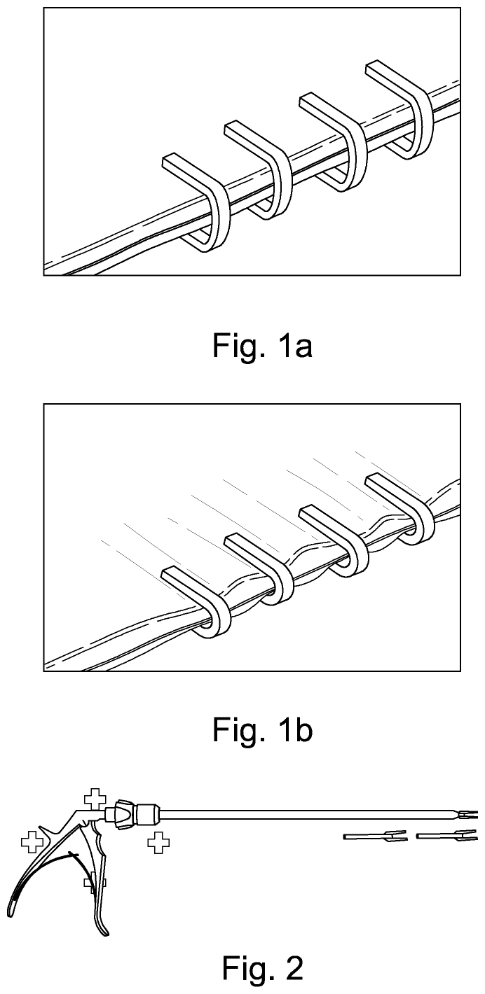

Whilst the primary intended function of a laparoscopic surgical fastener is to clamp fluid-carrying lumens, fasteners may be used for reinforcement or haemostasis of staple lines when bleeds occur (between staples or slow leaking through the line). Surgical fasteners, such as the ductile-clamping fasteners, shown in FIG. 1 are often used in this manner. When uncontrolled haemorrhage from staple line is observed during this type of surgery (in at least 75% of US cases) the standard solution is to apply several endoscopic fasteners.

However, despite the use of fasteners during surgery, reinforcement and haemostasis of staple line continues to present an issue in laparoscopic procedures. Such fasteners are not able to be accurately and/or neatly applied and are often clamped in an ad hoc manner resulting in an inconsistent application along the staple line. The applicants have recognised that such a solution is not deemed satisfactory by clinicians practising in the field.

Further, due to the difficulty of controlling the placement of the fastener an even pressure is not able to be applied and therefore leaking may continue where pressure is lowest along the tissue, leading to an undesirable surgical result and use of unnecessary additional fasteners.

Raney fasteners are typically used in neurosurgery to clamp the scalp as shown in U.S. Pat. No. 6,071,290 and allow a greater length of scalp to be clamped at once. During laparoscopic surgery conventional fasteners are loaded into the jaw which is longitudinal to the shaft of the instrument; this limits the width of the fastener to fit within the cross sectional width of the fastener applicator so that it can be passed through the trocar port. However, the Raney fastener is both cylindrical and elongate making it impractical as it would not fit through a trocar port.

The issues described above present in all different types of laparoscopic surgical procedure, for example, the closure of enterotomy, fistulas or perforations.

These problems are also observed regularly by clinicians during sleeve gastrectomy. Due to the increase in metabolic disorders and obesity, bariatric surgery is the fastest growing surgical specialty and sleeve gastrectomy is the bariatric operation most increasing in prevalence (Buchwald H, Oien D M. Metabolic/Bariatric Surgery Worldwide 2011. Obes Surg. 2013 Jan. 22). There were an estimated 340,768 bariatric procedures performed in 2011 in the UK alone, of which at least 30% were sleeve gastrectomies. Further, in this particular surgery there is evidence to suggest from meta-analysis that reinforcing staple line decreases overall complications (Choi Y Y, Bae J, Hur K Y, Choi D, Kim Y J. Reinforcing the staple line during laparoscopic sleeve gastrectomy: does it have advantages? A meta-analysis. Obes Surg. 2012 August; 22(8):1206-13).

The applicants therefore recognise that further solutions are required to provide an improved means for controlling bleeding and achieving a desirable surgical result particularly in laparoscopic surgery, such as bariatric surgery.

Surgical devices for applying the fasteners described above typically entail grasping or clamping tissue between opposing jaw structures and then joining the tissue by employing the fastener. A fastener applicator is loaded with the fastener and then manipulates it to lock together or deform. These general devices are well known in the art.

In the case of laparoscopic procedures, the fastener applicator is an instrument having a circular cross sectional profile, typically 5 or 10 mm in diameter, which can be inserted through the cannulas in the patient. Ductile fasteners, such as those referenced above, are typically of a ‘U’ or ‘V’ profile, are loaded into a jaw in the fastener.

Alternatively, fastener applicators may contain a magazine of surgical fasteners that can be fired in succession, in a similar manner to a stapler. Staplers typically comprise two elongated members which are respectively used to capture or clamp tissue. Typically, one of the members carries a cartridge which houses a plurality of staples arranged in at least two lateral rows while the other member comprises an anvil which defines a surface for forming the staple legs as the fasteners are driven from the cartridge. Where two part fasteners are used, this member carries the mating part, e.g. the receiver, to the fasteners driven from the cartridge. Generally, the stapling operation is effected by a pusher which travels longitudinally through the cartridge carrying member, with the pusher acting upon the staples to sequentially eject them from the cartridge. In fastener applicators, the fasteners are typically seated in a groove within the applicator jaws to prevent the fastener falling out or becoming misaligned. Pre-loading of the fastener into the jaw also helps prevent the fastener from falling out. The fastener is applied using a lever which actuates the jaws of the fastener applicator and compresses the fastener. A typical fastener applicator uses an inclined plane mechanism comprising of a compliant mechanism and a sleeve so the jaws of the fastener applicator are brought together as the sleeve is retracted over the compliant mechanism, such as the designs described in relation to the ductile fasteners above. A ratchet mechanism is typically used to prevent the applicator jaws inadvertently opening during the firing procedure if the operator releases the trigger midway through the fastener application.

Although reusable apparatuses have been developed their structure and configuration can be overly complex and prove difficult to sterilise. It is well known that there are dangers associated with improper sterilisation Re-sterilisable fastener applicators have been devised in order to reduce surgical consumable costs, such as the applicator described in U.S. Pat. No. 5,607,436. However, there remains a need for applicators which can be simple enough in their configuration and component structure to be easily re-sterilised or which provide a cost-effective disposable solution.

Further, where minimally invasive surgery is required (e.g. during laparoscopic procedures) articulating surgical fastener applicators have been devised in order to improve access to the surgical site. A typical fastener applicator designed to deploy a fastener for this use is provided in FIG. 2. However, providing suitable apparatus which improves deployment of surgical fasteners which, in turn, suitably control bleeding, as described in the before mentioned technical problem, continues to present a challenge. For example, clinicians find that laparoscopic fasteners must be applied at an oblique angle in order to cover the desired length of tissue, which makes clamping in a confined space very difficult.

Thus, due to the inherent spatial restrictions associated with surgical site access in laparoscopic surgery the applicant also recognises a need for an improved apparatus by which to insert, manipulate and deploy a fastener which reinforces the staple line and prevent haemorrhage.

### SUMMARY OF THE INVENTION

In one aspect, the invention concerns a surgical fastener having a first part and a second part, each of the first and second parts comprising a clamping bar and a centrally-positioned perpendicular bracing bar extending therefrom, wherein the clamping bar and bracing bar together defines a “T”-shape, or “T”-shaped profile, the fastener further comprising at least one bracing element connecting the respective bracing bar of each of the first and second parts.

In one embodiment the clamping bar is elongate. The longer elongate nature of the clamping portions of the fastener permits larger amount of tissue area to be clamped simultaneously which provides a more even pressure along the staple line, reduced leakage and thus improves haemostasis. Since the fasteners are able to cover a larger area of the tissue, fewer are needed to achieve a satisfactory surgical solution. Moreover, their simple design enables them to be produced through standard techniques known in the art which are cost effective. The fasteners of the invention therefore provide an economical solution, as well as a technical one.

Even though the fastener is, as a whole, elongate, it may be loaded in to the applicator by virtue of a fastener applicator having a complementary central T-junction support or recess structure that enables the fastener to be releasably retained in to the surgical cavity in one position or orientation during delivery to the surgical site and then rotated to a second different position or orientation. As a result, the elongate clamping bars of the fastener are then positioned substantially parallel to the tissue before being deployed First and second parts are preferably identical to ensure an even distribution of pressure above and below the tissue line.

In one embodiment the first and second parts each comprise a single bracing bar and a single bracing element. Only a single bracing feature is required, however the invention is not limited to this embodiment.

In a further embodiment there may be a plurality of bracings bars associated with each clamping bar. In a further preferred embodiment there are three bracing bars evenly spaced along each clamping bar of either or both the first and second parts. In one embodiment there is also a bracing element connecting together each pair of bracing bars provided on the respective first and second parts.

In one embodiment, one or more bracing element is curved. In one embodiment the bracing element comprises a plurality of minor elements to create a multi part bracing element which may allow the bracing element to deform in a predictable manner.

The fastener may be made of a ductile material. The material can be a medically-acceptable material that is ductile, malleable or plastically deformable (that is, any material that when bent remains in the bent condition) including stainless steel, titanium, tantalum and other non-bio absorbable and bio absorbable metal and plastic materials.

In one embodiment the bracing bar is made of a material which has a greater rigidity relative to another material or metal used for the bracing element. This allows the fastener to deform in a predictable and reliable manner.

In one embodiment the fastener further comprises a reinforcement feature. Such a feature may prevent deformation to the peripheral or far ends of the clamping bar of the fastener. In one embodiment the reinforcement feature is a reinforcement or stiffening flange which is formed along the outer edge perpendicular to the plane of the clamping bar and running the a substantial part of the length of the clamping bar. The fastener may additionally or alternatively include a groove etched between the flange and the clamping bar allowing the load applied to the fastener to be concentrated to an area which promotes desirable bending.

These reinforcing features allow the fastener to be clamped and retained on the tissue in a more reliable manner than achieved by conventional fasteners; the fastener deforms in a controlled manner and ensures that the fastener remains parallel with the staple line of the tissue while it is under load and deforms there around.

In a further embodiment, edges of elongate bar, bracing bar and/or reinforcement feature are curved and/or blunt. It is important that no feature of the fastener inherently or otherwise promotes ripping or cutting of the tissue or puts greater pressure on any part of the tissue which may result in tearing or causes bleeding.

In a preferred embodiment, in a non-deformed configuration (pre-clamping) the distance between the first part and second part of the fastener is approximately 5.5 mm.

In a preferred embodiment, the width of the bracing bar is less than the width of the clamping bar.

In a preferred embodiment, the width of the bracing bar is approximately 1 mm to 1.2 mm

In a particularly preferred embodiment, the first or second part and or the bracing bar or bracing element comprise an identifying feature which is associated with a fastener or part thereof having a particular dimension. In a preferred embodiment the identifying feature is a colour or text provided on the fastener or a part thereof.

In one embodiment the length of elongate bar is 8 to 24 mm. The particularly preferred length of elongate bar is no more the 50 mm.

In a further embodiment the depth of the clamping bar is approximately 0.6 mm.

In a further embodiment, the bracing bar comprises a mating element for operatively engaging with a corresponding mating element on a fastener applicator. For example, the mating element may be a recessed pivot formed in the bracing bar which permits the fastener to be releasably attached to the fastener applicator and be rotated by the applicator in the surgical space prior to clamping. The clamping motion disengages the mating elements thereby ensuring the fastener is released easily from the applicator. In a further embodiment more than one bracing bar of the fastener comprises a mating element.

In a further aspect the invention concerns a kit comprising a plurality of surgical fasteners for clamping tissue, each fastener being in accordance with the same or different embodiment defined above.

It is acknowledged that with a particularly preferred embodiment of the fastener being elongate and thus difficult to apply to tissue, at least one solution devised to utilise this fastener is required.

In a further aspect of the invention there is provided a surgical apparatus for the application of a fastener to tissue comprising: a handle assembly; a shaft extending distally from the handle assembly; a drive assembly; an articulation assembly; and a jaw assembly mounted adjacent a distal end of the shaft comprising fixed support jaw and a clamping jaw, wherein at least one of the jaws comprises a rotatable fastener support defining a T-shaped recess therein for receiving a surgical fastener, wherein the articulation assembly is operable to rotate the fastener support and the drive assembly to move the clamping jaw in and out of a closed configuration with respect to the support jaw. Typically the drive assembly and articulation assembly are operable by the handle assembly.

Usefully, the present invention therefore enables an elongated fastener of a corresponding T-shape, such as the arrangement of the fastener described herein above, to be loaded into the fastener support in a first longitudinal orientation such that it can be transported to the surgical site within the limitations of the normal endoscope/trocar and then rotated by the user of the apparatus about an angle between 0 and 90° into a second different orientation. The apparatus therefore provides that a fastener, which itself provides advantages inter alia compression along the staple line (by virtue of its elongate bar), can be easily manipulated and clamped about the staple line and, most preferably, can be clamped parallel to the staple line. Such a mechanism permits the fastener to be better controlled by the clinician in vivo, allowing for more accurate positioning and deployment resulting in an improved surgical result for the clinician and patient.

In one embodiment the support arm comprises a rotatable fastener support defining a T-shaped recess therein for receiving a surgical fastener. In a preferred embodiment, the fastener support(s) is generally annular, however it may comprise one flattened edge with a support side to better retain the elongate bar of the fastener during orientation.

In another embodiment both the support arm and clamping arm comprise a rotatable fastener support defining a T-shaped recess therein for receiving a surgical fastener

In one embodiment the articulation assembly comprises a pulley system including a groove formed in the one or more fastener supports and corresponding antagonistic tendons operatively associated with the handle assembly via the shaft such that the handle assembly is operable by a user to rotate the one or more fastener supports. In particularly preferred embodiments, the groove is annular and/or the pulley system is spring-loaded to retain tension in the tendons.

The surgical apparatus may further comprise a dial, button or interface on the handle assembly by which the user may operate the articulation assembly. In one embodiment the handle assembly comprises a thumbwheel operable with the articulation assembly to rotate the fastener when situated in the fastener support.

In an alternative aspect there is provided a surgical apparatus for the application of a fastener to tissue comprising: a handle assembly; a shaft extending distally from the handle assembly; a drive assembly; an articulation assembly; and a jaw assembly mounted adjacent a distal end of the shaft comprising fixed support jaw and a clamping jaw, wherein at least one of the jaws comprises a fastener engaging member for rotating the fastener wherein the articulation assembly is operable to rotate the fastener and the drive assembly is operable to move the clamping jaw in and out of a closed configuration with respect to the support jaw. Such an arrangement therefore provides an alternative solution to rotating a fastener that does not necessarily have a complementary recess to fit the fastener that is to be retained and rotated.

In a further embodiment, at least one of the jaws further comprises a fastener support for releasably securing a surgical fastener thereto. In another embodiment, the fastener engaging member also releasably secures the surgical fastener thereto.

In a further aspect there is disclosed a surgical apparatus for the application of a fastener to tissue comprising: a handle assembly; a shaft extending distally from the handle assembly; a drive assembly; and a jaw assembly mounted adjacent a distal end of the shaft comprising fixed support jaw and a clamping jaw, wherein at least one of the jaws comprises fastener support defining a T-shaped recess therein for receiving a surgical fastener therein, wherein the drive assembly is operable by the handle assembly to move the clamping jaw in and out of a closed configuration with respect to the support jaw. In such a case, it may not be necessary to utilise a fastener with a clamping bar which extends beyond the circumference of the transporting mechanism to the surgical site. In other words, if such a fastener requires a T-shape as defined by the above description but is not elongate (such that requires necessarily to be rotated at the surgical site) then the apparatus may simply have a correspondingly-shaped T-shape recess within at least one of the two jaw members with which it is able to releasably receive the fastener and clamp it in position.

In one embodiment, the surgical apparatus of the invention may be disposable. However, it is noted by the inventors that development of the present invention has resulted in a relatively simple configuration which enables the components of the apparatus to be few and non-complex in their arrangement. The components of the fastener applying apparatus therefore may be reusable. In one such embodiment the apparatus of the invention as defined above facilitates proper sterilisation after use in a surgical procedure and can be re-used thereafter.

### BRIEF DESCRIPTION

Various features, embodiments and examples of the presently disclosed surgical fastener and fastener applying apparatus will now be described herein with reference to the accompanying figures wherein:

FIG. 1a is a perspective view of an unclamped ductile fastener of the prior art;

FIG. 1b is a perspective view of a clamped ductile fastener of the prior art;

FIG. 2 is a perspective view of a surgical fastener applier of the prior art;

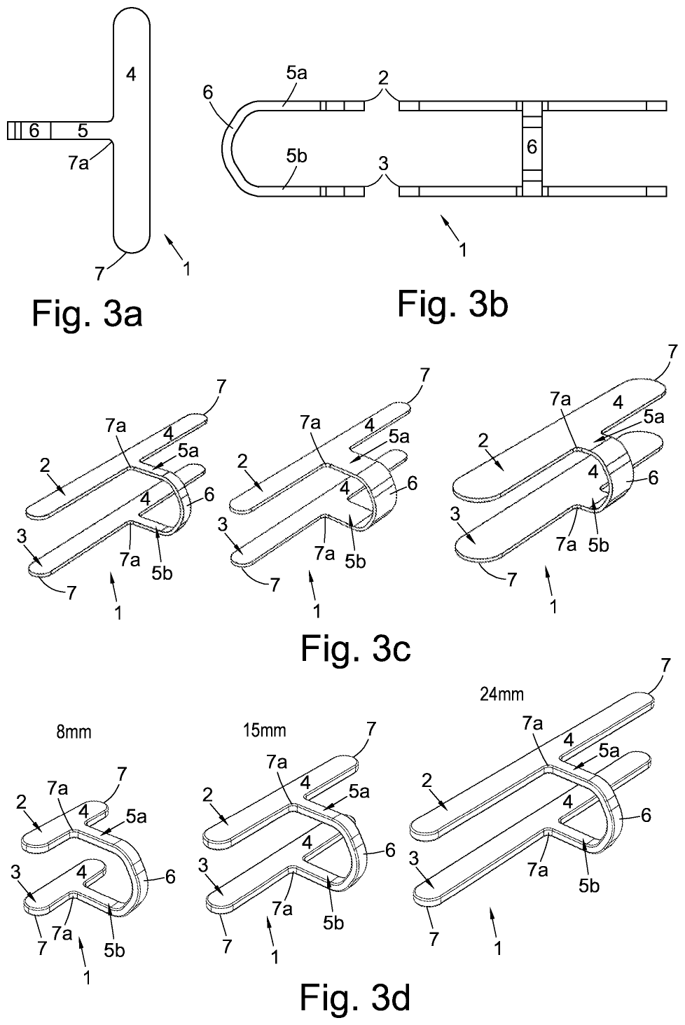

FIG. 3a is a is a top view of one embodiment of the T-shaped fastener of the invention;

FIG. 3b shows side views of one embodiment of the T-shaped fastener of the invention;

FIG. 3c is a perspective view of alternative embodiments of the T-shaped fastener of the invention;

FIG. 3d is a perspective view of yet further alternative embodiments of the T-shaped fastener of the invention;

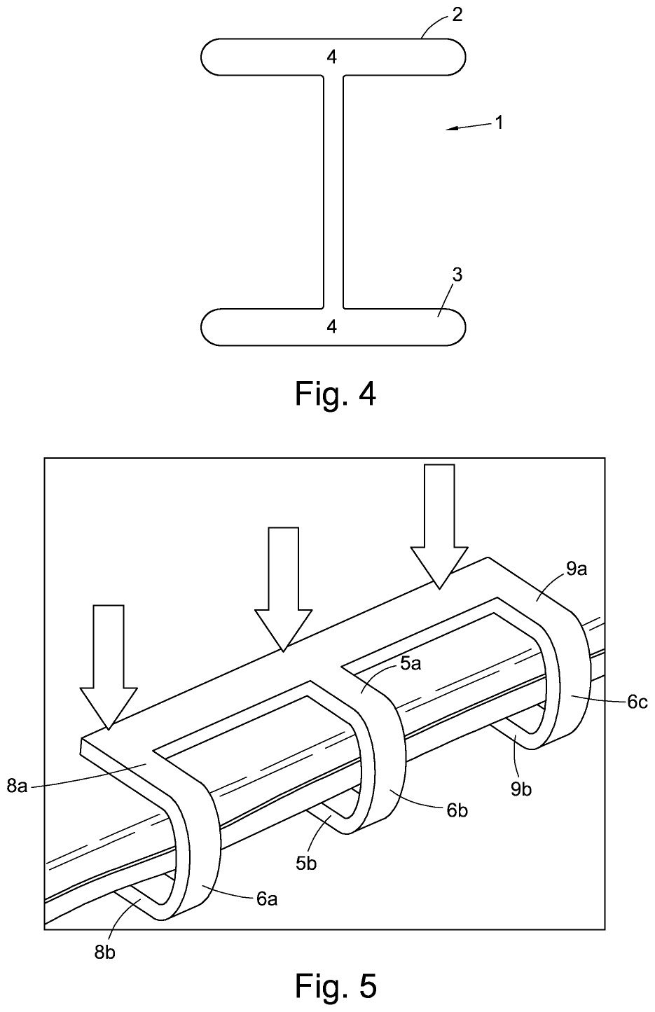

FIG. 4 is a view of the T-shaped fastener having been pressed out during manufacture and prior to final formation for use;

FIG. 5 is a perspective view of a further embodiment in which the T-shaped fastener of the invention which has a multi-bracing;

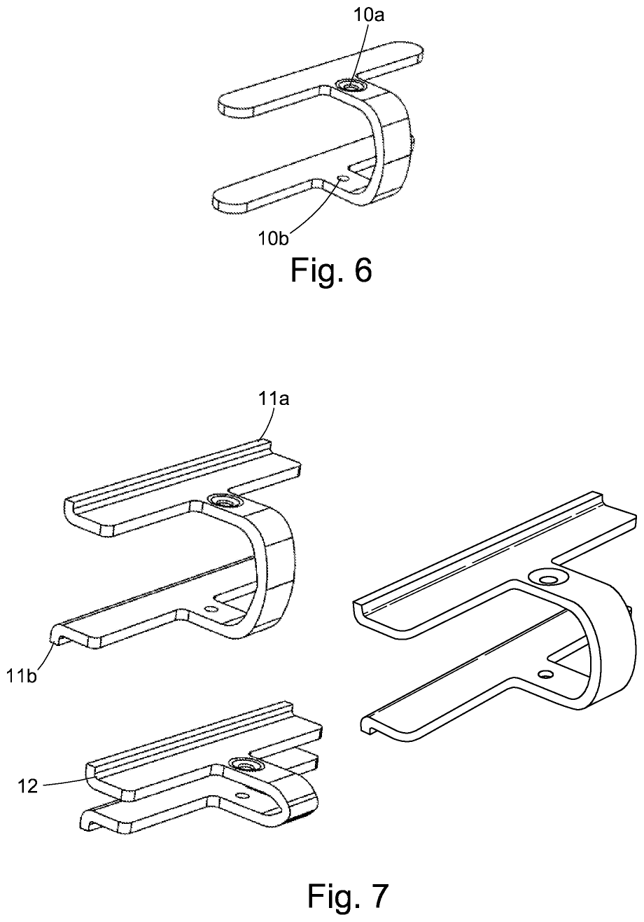

FIG. 6 is a perspective view of a further embodiment of the T-shaped fastener of the invention;

FIG. 7 is a perspective view of yet a further embodiment of the T-shaped fastener of the invention;

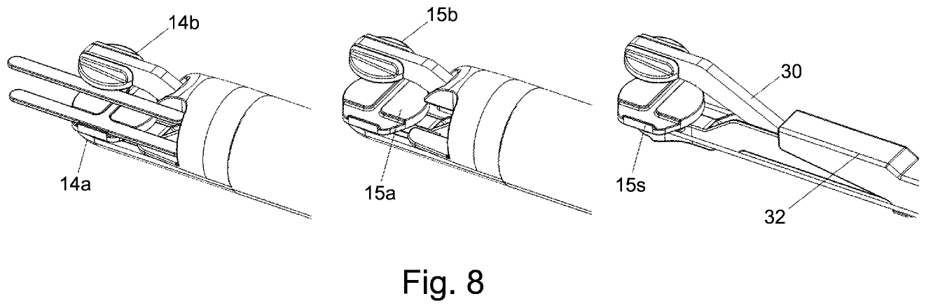

FIG. 8 provides perspective views of a distal end of an embodiment surgical fastener applier in use;

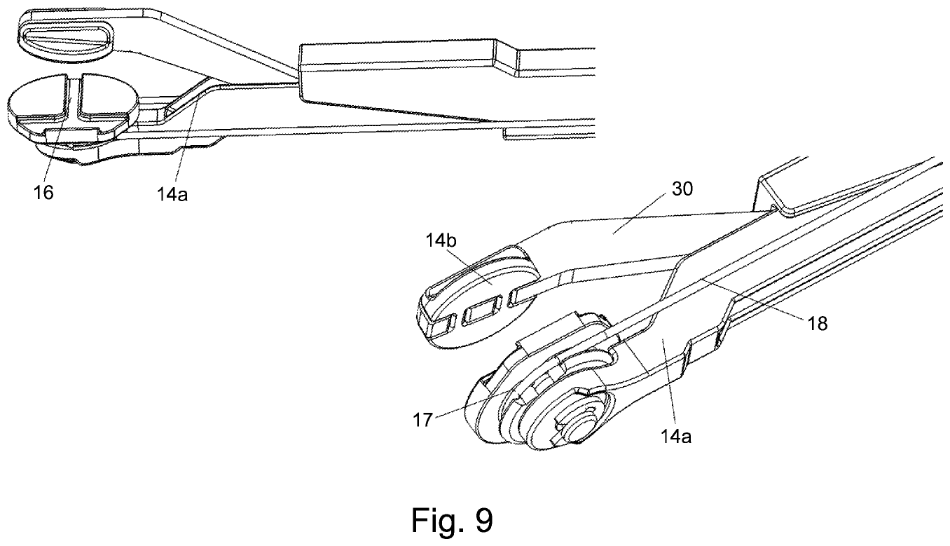

FIG. 9 provides perspective views of the jaw assembly of the surgical apparatus of FIG. 8;

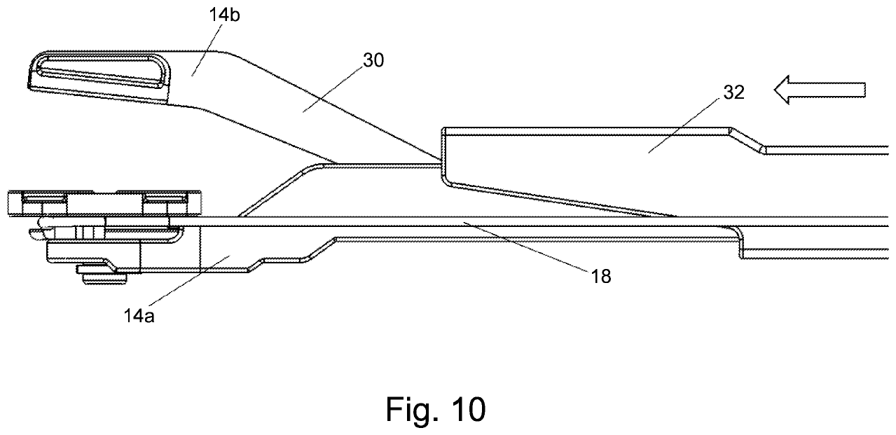

FIG. 10 provides a side view of the jaw assembly of the surgical apparatus of FIG. 8;

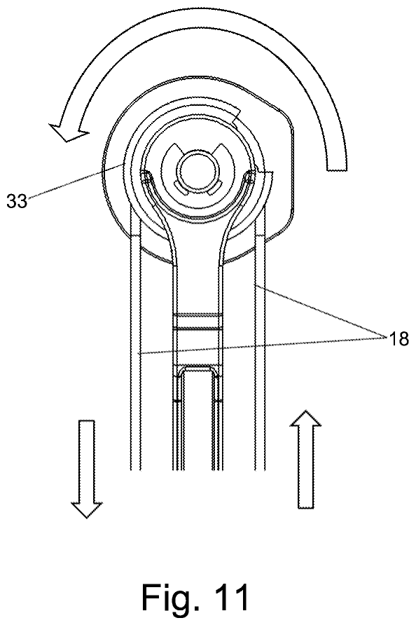

FIG. 11 provides a top view of part of the internal articulation assembly according to an embodiment of the surgical fastener applier;

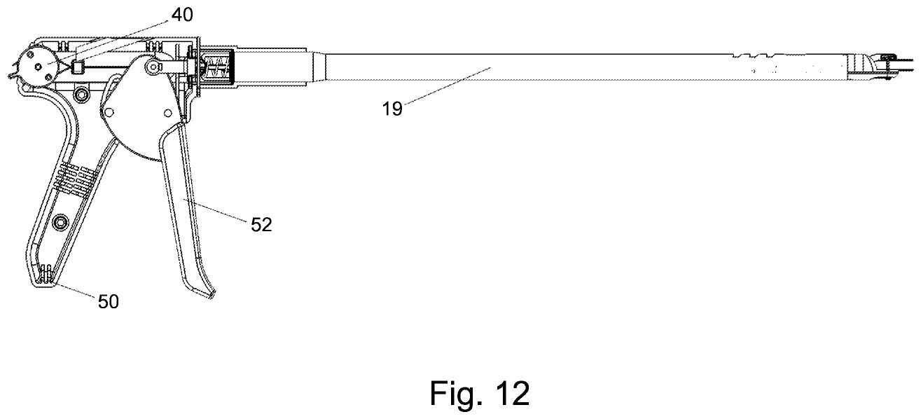

FIG. 12 provides a side view of a surgical fastener applier according to an embodiment of invention;

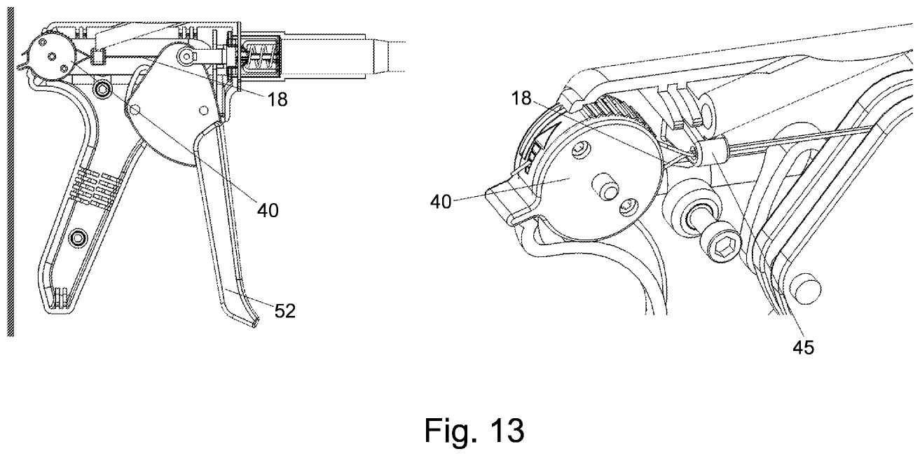

FIG. 13 provides internal side and perspective views of the components of a handle assembly of the surgical fastener applier in accordance with an embodiment of the invention;

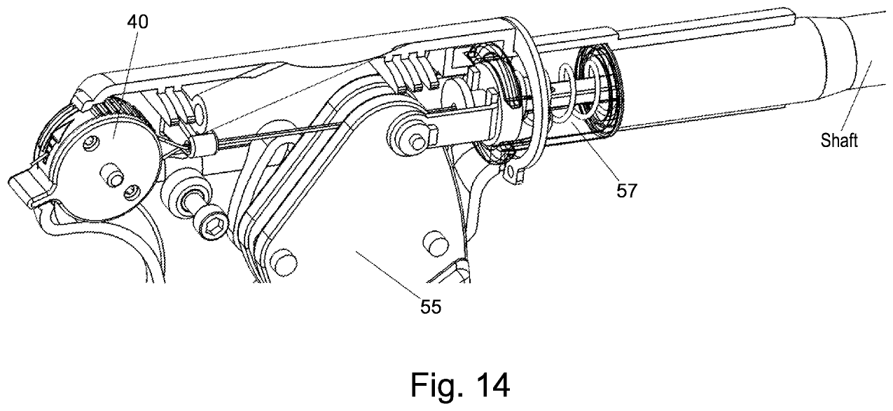

FIG. 14 provides an internal perspective view of the handle assembly and articulation mechanism assembly of the surgical fastener as shown in FIG. 13.

### DETAILED DESCRIPTION

As used herein, the term “proximal” refers to the end of the apparatus or feature which is closer to the user or clinician and the term “distal” refers to the end of the apparatus which is further away from the user.

FIG. 1a is a perspective view of a plurality of unclamped ductile fasteners of the prior art. Such fasteners generally each comprise a single U-shaped body, which extends either side of the tissue to be clamped. As can be seen in perspective view FIG. 1b, the fasteners of the prior art are in a clamped configuration wherein an uneven pressure is provided along the upper and lower surfaces of the staple line, creating a dimpling effect and resulting in pockets through which blood may leak.

FIG. 2 is a perspective view of a surgical fastener applier of the prior art comprising a proximal handle, shaft and clamping end effector at a distal end of the shaft, the end effector comprising jaws for receiving and clamping a clip, wherein the jaws are operably linked to the handle. The user operates the handle to drive the jaws together moving the legs of the clip toward one another into a closed configuration around the tissue. The whole shaft may be rotated by the user by twisting a standard annular grip which surrounds the shaft adjacent the handle.

Embodiments of the surgical fastener and surgical fastener applying apparatus in accordance with the present invention will now be described in detail with reference to the drawings.

Referring now to FIGS. 3a, 3b and 3c and 3d, a surgical fastener or clip 1 generally has a first part 2 and a second part 3, each of the first and second parts comprising an elongate clamping bar 4, which may be a flattened plank. In the same plane, for each clamping bar there extends a bracing bar 5, 5a, 5b, which extends perpendicularly to the elongate clamping bar and is typically centrally positioned along its length.

From a top view in FIG. 3a it can be clearly seen that clamping bar 4 and bracing bar 5 together define a distinctive “T”-shape.

It can be seen in FIGS. 3a-d that the fastener further comprises at least one bracing element 6 which joins the first and second parts 2, 3 of the fastener via their respective ends of the bracing bars 5a, 5b. In particular, the bracing element 6 of the fastener may be integrally moulded to form a humped curve defining multi-section element, as shown in FIGS. 3b, c and d.

The construction of the fastener functioning as a T-shaped clamp is fundamental to providing balanced distribution of pressure on to the tissue when clamping the tissue during surgery. It also enables clamping over a sufficiently large surface area to reduce the likelihood that the fastener will move or slip in a lateral or longitudinal direction along the tissue. Further, in embodiments where the fastener has identical first and second parts 2 and 3 this further ensures an even distribution of pressure when clamping tissue there between.

Further, by virtue of first and second parts having an elongate clamping bar 4 a longer portion of tissue extending along the staple line can be clamped with even pressure, minimising the number of fasteners required and shortening the time needed to effectively minimise bleeding at the staple line.

As can be seen in FIGS. 3a, c and d the external edges 7 of the elongate members of both first and second parts 2, 3 are either curved and/or blunt to ensure the risk of perforation of the tissue is minimised when the fastener is clamped. Furthermore, for the same reason, the internal edges 7a either side of the bracing bars 5a and 5b joining the internal edge 7a of the clamping bar 4 of first and second parts 2, 3 may also be curved and or smooth.

The clamping bar may be sized such that the particular fastener is useful for certain surgical applications. FIG. 3c shows alternative fasteners in which the width of the clamping bar and/or bracing bar is increased or reduced in accordance with such applications.

It is not essential that the clamping bar is elongate. Where the clamping bar is not elongate it is preferred that the size of the clamping bar is defined by the maximum width of the applicator by which it is inserted and deployed; such an example is described in use with one form of the apparatus further below. Essentially, a constant and balanced pressure can be obtained when the fastener of this definition clamps a section of tissue together. A non-elongate embodiment is shown in FIG. 3d as compared to elongate examples wherein the clamping bar is elongate and the length of the overall fastener has been increased. Additionally the depth or thickness of the whole fastener may be increased dependent of the particular surgical application (also shown in FIG. 3d). An embodiment with increased thickness permits the fastener to be further shaped during construction to remove any sharp edges its final form, preventing completely the possibility of sharp edges contacting the tissue surface during use.

FIG. 5 shows a further example of the invention in which each fastener has three bracing elements 6a, 6b, 6c, wherein first and second parts 2, 3 each have a clamping bar 4 with three corresponding bracing bars 8a, 5a, 9a and 8b, 5b, 9b, respectively. This arrangement of multiple bracings allows for a greater degree of clamping force to be exerted evenly across the tissue and would further prevent the fastener from slipping and occurrence of leakage along the staple line.

In an alternative embodiment of the fastener, such as the arrangement shown in FIG. 6, there may be a recess 10a, 10b machined in to the bracing bar of one or both of the first and second parts 2, 3.

One recess 10b assists the fastener to be releasably retained in the jaw of a fastener applying apparatus during loading and transfer of the fastener to the surgical site, for example, by engaging with a corresponding retaining feature positioned in the jaw. The other recess 10a may assist rotation of the fastener by, for example, co-operating with or engaging a corresponding mating feature positioned in a jaw of the fastener applying apparatus which is operable to rotate the fastener.

Embodiments of the invention may comprise one or both recesses. The fastener may be easily released at the surgical site since the clamping action by the fastener applying apparatus compresses the first and second parts together, thereby moving one or both recesses out of registration with the retaining feature or mating feature on the respective jaws of the apparatus.

FIG. 7 shows a perspective view of a clamped and unclamped embodiment of the fastener of the invention with, for example, the recesses described above in FIG. 4. In addition the fastener may further comprise reinforcement flanges or stiffening ledges or the like 11a, 11b, formed from the outer edge of the respective clamping bars 4 and running the length of the clamping bar.

Reinforcement of the clamping bar may assist in preventing potential deformation to the peripheral or far ends of the clamping bars of the fastener after clamping. The flanges 11a, 11b extend in opposing directions each perpendicular to their respective clamping bars. The flanges may be formed integrally with the clamping bars each forming a smooth curved surface area together with the clamping bars such that no edges of the clamping bars dig in to tissue when pressure is applied as they are clamped together. In particular, the edges of elongate bars, bracing bars and/or reinforcement features are curved and/or blunt. It is important that no feature of the fastener inherently or otherwise promotes ripping or cutting of the tissue or puts greater pressure on any part of the tissue which may result in tearing or otherwise cause bleeding.

Further as can be seen labelled in the clamped orientation of the fastener, additionally or alternatively to the reinforcement feature, there may include a groove 12 etched between the flanges and the clamping bars allowing the load applied to the fastener to be concentrated to an area which promotes desirable bending of the fastener with reduced force.

These reinforcement features allow the fastener to be clamped and retained on the tissue in a more reliable manner than achieved by conventional fasteners; the fastener deforms in a controlled manner and ensures that the fastener remains parallel with the staple line of the tissue while it is under load and deforms there around.

The additional reinforcement features of this example may be provided with or without recesses in preferred embodiments.

The parts of the fastener described herein above are typically formed integrally i.e. formed as a whole. The fastener of the invention may be manufactured by forming a shape as disclosed in any of the herein described figures.

Accordingly, the fastener may first be stamped, forged, moulded, or machined into a first desired shape, for example as shown in FIG. 4, from a ductile material or metal, such as Titanium, for example, ASTM F67 grade 1. The material must be able to deform without returning to its original shape or fracturing under pressure. However, the fastener may be formed from other materials suitable for use in surgery such as alloys, steel, metal, other grades of titanium, or other biocompatible or suitable implantable material.

Different parts of the fastener may be made from different materials to impart certain characteristics to the fastener. For example, the bracing bar can be made of a material which has a greater rigidity relative to metal used for forming the bracing element. This allows the fastener to deform in a more predictable and reliable manner.

To form the fastener into a shape ready for loading, the fastener is treated in accordance with typical annealing methods known in the art. The fastener may be heated in a vacuum. Thereafter, once the fastener is bent or folded into its form it may be cooled slowly, in accordance with typical techniques known in the art. Such steps may be varied in accordance with known heat treatment and cooling processes in order to stress relieve and change the fastener hardness and strength of the final fastener.

Variations of the presently disclosed fastener shapes are possible and within the scope of the present disclosure provided the major features of the invention are present, as defined by the claims.

Description of loading and applying various fasteners using different versions of apparatus, as required, will now be described below;

FIG. 8 shows the distal end portion of the apparatus of the invention for applying T-shaped fasteners.

The apparatus comprises general features typically well known in the art including a proximal end portion (not shown) comprising a handle assembly with a grip and a lever operable to close jaws of the apparatus. The apparatus further comprises an outer body and longitudinal axis defined there along (not shown). The outer body is an elongated shaft which extends between the proximal end and the distal end of the apparatus and includes an axial passage there between. The shaft and handle may be made from a variety of rigid biocompatible materials, such as stainless steel, a plastic such as acrylonitrile-butadiene-styrene (ABS), a polycarbonate material or any other biocompatible material capable of the requirements described herein. In a preferred embodiment, the shaft is rigid; however it may also be made from a semi-compliant or flexible material to allow some flexibility along the axis. The shaft is tubular and has a relatively small outer diameter for insertion into and through a trocar cannula or other small incision. The outer diameter of the shaft is limited in size by the inner diameter of the trocar through which it must pass to access the surgical site.

The distal end of the apparatus comprises a two jaw assembly mounted adjacent a distal end of the shaft comprising fixed support jaw and a clamping jaw 14a, 14b. A drive assembly is operable by the handle assembly to move the clamping jaw in and out of a closed configuration with respect to the support jaw by methods known well to those skilled in the art. This is typically achieved through an elastically deflecting clamping jaw and an inclined plane mechanism. The elastically deflecting clamping jaw has an inclined feature 30 so that when a sliding component 32 traverses across, a force is imparted perpendicular to the plane, so as to cause deflection of the cantilevered jaw 14b.

Any other mechanical means of rotating the clip in the jaw would have to compensate for the deflection of the jaw in a dual clamping jaw; hence, it is practical to have a fixed jaw and a moving/clamping jaw.

In the example, the fixed jaw further includes a rotatable fastener support 15a comprising a T-shaped recess 16 for receiving a fastener 1 having a corresponding configuration therein. Each fastener support is generally annular in shape but may have one flattened side 15s to facilitate its enclosure within the assembly as a whole and support and manoeuvre the fastener more securely.

In FIG. 8 the apparatus is shown in a non-rotated position in which an elongate T-shaped fastener has been inserted into the T-shaped recess 16 of the fastener support 15a and orientated longitudinally therewith.

As can be seen in FIG. 9, the rotatable fastener support may further comprise an annular groove 17.

The apparatus also comprises an articulation assembly which includes a pulley system comprising antagonistic tendon 18 arranged about the annular groove 17 of a pulley 33 associated with the rotatable fastener support. The articulation assembly operates to rotate the fastener support as shown in FIG. 11.

The antagonistic tendon may be attached to an actuating mechanism using any of a variety of methods available and known to those of skill in the art. As an example, the proximal end of a tendon may be attached to the actuating mechanism which in turn is operably linked by manipulating a thumb wheel 40 as shown in FIGS. 11-14. Alternatively, this could be any rotatable feature such as dial, button or the like on the handle assembly which is operable to move antagonistic tendons 18 and rotate the fastener support

In FIGS. 11-14 the fastener support is rotated by an antagonistic tendon system whereby the tendons 18 are terminated at the pulley through a ferrule or using a grub screw or a plastically deformable feature that grips the tendon to prevent slippage when tension is applied, and thus apply torque to the pulley. The tendons may run through a low friction sleeve 45, such as in a Bowden cable arrangement, or be passed through a low friction bearing surface within the shaft of the instrument.

In an alternative design, there is only a single pull tendon and a spring is used to return the pulley to the 0° position. However, other methods may be used to articulate the fastener support in order to rotate the fastener from an orientation in which it is longitudinally aligned with apparatus to a second orientation where it has been rotated from 10 to approximately 90 degrees.

Once the fastener support and thus fastener within has been rotated by the desired amount a drive assembly is operable by the handle assembly to move the clamping jaw in and out of a closed configuration with respect to the support jaw by methods known well to those skilled in the art.

FIG. 12 shows an embodiment of the apparatus utilised to retain, insert, rotate and clamp a fastener in vivo. The apparatus comprises general features typically well known in the art for clamping a fastener including a proximal end portion comprising a handle assembly with a grip 50 and a lever 52 operable to close jaws of the apparatus. The apparatus further comprises an outer body and longitudinal axis defined there along. The outer body is an elongated shaft 19 which extends between the proximal end and the distal end of the apparatus and includes an axial passage there between. The shaft is tubular and has a relatively small outer diameter for insertion into and through a trocar cannula or other small incision. The outer diameter of the shaft is limited in size by the inner diameter of the trocar through which it must pass to access the surgical site. The whole shaft may also be rotated by the user by twisting a standard annular grip which surrounds the shaft adjacent the handle.

As shown in FIGS. 13 and 14 the proximal end of the apparatus comprises a trigger or lever linkage 55 to translate movement when the user pulls the lever towards the grip 50 in order to drive the clamping jaw towards the fixed jaw. The drive assembly may further include a return springe 57 to urge the lever back to its original position after being operated by the user.

In some embodiments, where the T-shaped fastener is not elongate (e.g. see the smallest of fasteners in FIG. 3d), a rotation mechanism is unnecessary and therefore the invention extends to embodiments in which no articulation mechanism for rotation is required.

However, in preferred embodiments an elongate T-shaped fastener of the invention is utilised with said apparatus and therefore an embodiment of the apparatus which may easily insert the elongate fastener and has the function to rotate said fastener once in vivo has multiple benefits in surgery, as explained previously.

In other embodiments, instead of a T-shaped recess and rotatable support, the fixed jaw has a fastener engaging member for rotating the fastener wherein the articulation assembly is operable to rotate the fastener via the engaging member (and the drive assembly is operable to move the clamping jaw in and out of a closed configuration with respect to the support jaw). It is envisaged that such an embodiment would be utilised with a fastener as shown, for example, in FIG. 6 or 7 where the fastener has a corresponding means, such as a recess, to engage, at least temporarily with the surgical applier.

1. A surgical fastener having a first part and a second part, each of the first and second parts comprising a clamping bar and a centrally-positioned perpendicular bracing bar extending therefrom, the clamping bar being elongate relative to the bracing bar; the clamping bar and bracing bar together defining a “T”-shape,
wherein the surgical fastener further comprises at least one bracing element connecting the bracing bar of each of the first and second parts and
wherein the surgical fastener is, as a whole, elongate.

2. The surgical fastener of claim 1, wherein first and second parts are substantially identical.

3. The surgical fastener of claim 1 or claim 2, wherein there is a plurality of bracings bars associated with each clamping bar, preferably wherein there are three bracing bars evenly spaced along each clamping bar of either or both the first and second parts and more preferably a bracing element connecting the bracing bars of the first and second parts.

4. The surgical fastener of any preceding claim, wherein the at least one bracing element is curved.

5. The surgical fastener of any preceding claim, wherein the bracing element comprises a plurality of elements to create a multi-part bracing element.

6. The surgical fastener of any preceding claim, wherein the bracing bar is made of a material which has a greater rigidity relative to material used for forming the at least one bracing element.

7. The surgical fastener of any preceding claim, wherein the surgical fastener further comprises a reinforcement feature, preferably wherein the reinforcement feature is a reinforcement or stiffening flange

8. The surgical fastener of any preceding claim wherein the tissue contacting edges of elongate bar, bracing bar and/or reinforcement feature are curved and/or blunt.

9. The surgical fastener of any preceding claim wherein, the bracing bar comprises a mating element for operatively engaging with a corresponding element on a fastener applicator.

10. A kit comprising a plurality of the surgical fastener as defined by one or more of the preceding claims.

11. A surgical apparatus for the application of a fastener to tissue comprising:
a handle assembly;
a shaft extending distally from the handle assembly;
a drive assembly;
an articulation assembly; and
a jaw assembly mounted adjacent a distal end of the shaft comprising fixed support jaw and a clamping jaw, at least one of the jaws comprises a rotatable fastener support defining a T-shaped recess therein for receiving a surgical fastener,
wherein the articulation assembly is operable to rotate the fastener support and the drive assembly is operable by the handle assembly to move the clamping jaw in and out of a closed configuration with respect to the support jaw.

12. The surgical apparatus of claim 11, wherein the fastener support(s) is generally annular, preferably with one flattened edge.

13. The surgical apparatus of claim 11 or 12, wherein both the support arm and clamping arm comprise a rotatable fastener support defining a T-shaped recess therein for receiving a surgical fastener.

14. The surgical apparatus of claims 11 to 13 wherein, the articulation assembly comprises a pulley system including a groove formed in the one or more fastener supports and corresponding antagonistic tendons operatively associated with the handle assembly via the shaft, preferably wherein the groove is annular and/or the pulley system is spring is loaded to retain tension in tendons.

15. The surgical apparatus of any preceding claim, wherein the handle assembly comprises a thumb wheel, dial, button or interface for operating the articulation assembly.

16. A surgical apparatus for the application of a fastener to tissue comprising:
a handle assembly;
a shaft extending distally from the handle assembly;
a drive assembly;
an articulation assembly; and
a jaw assembly mounted adjacent a distal end of the shaft comprising fixed support jaw and a clamping jaw, at least one of the jaws comprising a fastener engaging member for rotating the fastener wherein the articulation assembly is operable to rotate the fastener and the drive assembly is operable to move the clamping jaw in and out of a closed configuration with respect to the support jaw.

17. The surgical apparatus of claim 16, wherein at least one jaw comprises a fastener support for releasably securing a surgical fastener thereto.

18. A surgical apparatus for the application of a fastener to tissue comprising:
a handle assembly;
a shaft extending distally from the handle assembly;
a drive assembly; and
a jaw assembly mounted adjacent a distal end of the shaft comprising fixed support jaw and a clamping jaw, at least one of the jaws comprising fastener support defining a T-shaped recess therein for receiving a surgical fastener therein,
wherein the drive assembly is operable by the handle assembly to move the clamping jaw in and out of a closed configuration with respect to the support jaw.
